(19) United States
(12) Patent Application Publication
(10) Pub. No.: US 2010/0069308 A1
(43) Pub. Date: Mar. 18, 2010

(54) Surfaces containing antibacterial polymers

(76) Inventors: IIya Chorny, San Francisco, CA (US); Kenneth Austin Dill, Montara, CA (US)

Correspondence Address: Ilya Chorny CEO, Simprota Corporation, 1700 4th St., Suite 218E, MC2522, San Francisco, CA, 94107, US

(21) Appl. No.: 12/455,817

(22) Filed: Jun. 8, 2009

Related U.S. Application Data
(60) Provisional application No. 61/059,316, filed on Jun. 6, 2008.

(51) Int. Cl. A61K 38/08; A61P 31/04

(52) U.S. Cl. 514/16; 514/17

(57) ABSTRACT

This invention provides a surface that has been coated with anti-bacterial polymers. Such surfaces would be suitable for use in, for example, medical devices and public surfaces. Additionally, the invention provides a method for coating surfaces with the anti-bacterial polymer.

### CROSS REFERENCE TO RELATED APPLICATION

This application claims the priority benefit of U.S. Provisional Patent Application No. 61/059,316 filed on Jun. 6, 2008 and is hereby incorporated by reference as if fully put forth below.

### FIELD OF THE INVENTION

This invention relates to surfaces containing an anti-bacterial polymer, more particularly, an anti-bacterial polymer comprising one or more n-substituted glycine monomers. More particularly, it includes embodiments where the anti-bacterial polymer is positively charged and amphiphilic and where the polymer shows antibacterial activity.

### BACKGROUND OF THE INVENTION

A major biomedical problem is the emergence of harmful bacteria that are resistant to modern anti-bacterial drugs, such as Methicillin-resistant Staphylococcus aureus (MRSA). These are often referred to as “superbugs.” There is a growing number of drug-resistant strains of bacteria; there are increasing numbers of hospital acquired infections. The problem arises because most antibacterial molecules are designed to act along specific biochemical pathways within a bacterium, and bacteria simply find new evolutionary solutions by creating alternative biochemical pathways around the drug's blockage mechanism. A better solution would involve disrupting the bacterial membrane, because bacteria have no evolutionary alternative to membrane encapsulation. A highly active research area involves this kind of approach to antibacterials, based on peptide molecules, which insert into membranes, poking holes in them and killing the bacteria by osmotic shock. Naturally occurring antibacterial peptides include defensins, indolicidin, magainin, pexiganan, and melitin. U.S. patent application Ser. No. 11/368,086, filed on Mar. 3, 2006, hereby incorporated by reference as if fully put forth below, contains additional examples of antibacterial peptides.

In addition to the emergence of “superbugs”, more than 40% of all hospital-acquired infections are associated with medical devices, such as catheters. Accordingly eliminating device associated infections should greatly decrease the number of hospital acquired infections. Strategies for coating medical devices include using silver and antibiotic formulations to coat the surface. One drawback to using silver and antibiotics is that the mechanism of action requires that they leach from the surface. Additionally, both silver and antibiotics can induce unwanted resistance and stimulate the evolution of additional “superbugs”. A novel approach is to coat medical device surfaces with covalently attached antibacterial peptides, as described in U.S. patent Ser. No. 11/675,500, filed on Feb. 15, 2007, hereby incorporated by reference as if fully put forth below.

Due to their mechanism of action, such antibacterial peptides should not be susceptible to resistance. However one drawback of using peptides, as demonstrated by our own internal data, show in FIG. 1, is that peptides become inactivated in the presence of biological fluids. In FIG. 1, panels 100A and 100B show the number of bacterial colonies as a function of the concentration of pexiganan 101A/B and ID 1 103 A/B, after 2 and 24 hours respectively, in biological serum. After 24 hours (100B) the pexiganan 101B no longer shows activity while ID 1 (103B) retains activity. Accordingly, a peptide alternative that kills bacteria by a mechanism similar to that of antibacterial peptides but without the biological susceptibility would provide greater protection against bacterial colonization.

Additionally, peptide bonds are susceptible to acid and/or base degradation, thus making peptides sub optimal as an antibacterial ingredient in cleaning solutions or in highly acidic or basic environments.

N-substituted glycines, or peptoids, are another class of foldameric polymers (polymers which form secondary structure) that have been shown to have anti-bacterial properties. A recent publication by Chongsiriwatana et al. “Peptoids that mimic the structure, function, and mechanism of helical antimicrobial peptides”, PNAS (2008) vol. 105:2794-2799, hereby incorporated by reference as if fully put forth below, demonstrates that peptoids can be designed with activities which are better than or equal to their peptide counterparts. In addition, peptoids offer the advantage that they are not susceptible to protease or acid/base degradation.

### SUMMARY OF THE INVENTION

This invention provides a surface that has been coated with anti-bacterial polymers. Such surfaces would be suitable for use in, for example, medical devices and public surfaces. Additionally, the invention provides a method for coating surfaces with the anti-bacterial polymer.

Further, this invention describes a surface coated with an antibacterial molecule, wherein the anti-bacterial molecule is an anti-bacterial polymer comprising one or more n-substituted glycine monomers, and wherein the polymer shows antibacterial activity.

In addition, the anti-bacterial polymer may be a chimeric polymer comprising amino-acid and n-substituted glycine monomers.

In addition, the anti-bacterial polymer may be covalently attached to the surface.

In addition, the anti-bacterial polymer may be non-covalently attached to the surface.

In addition, the anti-bacterial polymer may be positively charged and amphiphilic.

In addition, the anti-bacterial polymer can be selected from group comprising ID 1-19.

Further, the current invention describes a method for creating a surface having antibacterial activity comprising applying an anti-bacterial polymer to the surface, wherein the anti-bacterial polymer includes at least one n-substituted glycine monomer and wherein the anti-bacterial polymer has antibacterial activity.

In addition, applying the anti-bacterial polymer may comprise covalently attaching the polymer to the surface.

In addition, applying the anti-bacterial polymer may comprise non-covalently attaching the polymer to the surface.

In addition, the anti-bacterial polymer may be selected from a group comprising ID 1-19.

Further the current invention teaches that the anti-bacterial polymer may be grafted to the surface using a grafting polymer. The grafting polymer may include any polymer having self-adsorbing properties. The grafting polymer may be copoly(DMA-NAS-MAPS) as described below.

Further the invention describes covalently attaching the anti-bacterial polymer to Star PEG. In one preferred embodiment, the star PEG is covalently attached to the surface. In another embodiment, the star PEG is adsorbed to the surface

Further, the invention describes a linker positioned between the anti-bacterial polymer and the surface or the grafting polymer, wherein the linker is covalently attached to the anti-bacterial polymer. The linker may be a polymer comprising 1-1000 n-substituted glycine monomers where the substitution is with an Nme side chain. In one preferred embodiment, the linker comprises 10 Nme monomers. In another preferred embodiment, the linker comprises 20 Nme monomers.

Further, the invention describes a paint, resin cleaning solution, or analogous surface coating comprising the antibacterial polymer as an antibacterial ingredient, wherein the antibacterial polymer is added to prevent bacterial or fungal growth on a surface treated with paint, resin, or a cleaning solution containing the antibacterial material. In this embodiment the antibacterial polymer may be directly added as an ingredient without covalent attachment to the paint, resin, or cleaning solution.

### BRIEF DESCRIPTION OF THE DRAWINGS

FIG. 1 depicts the activity of ID 1 and an antibacterial peptide (pexiganan) in the presence of biological serum.

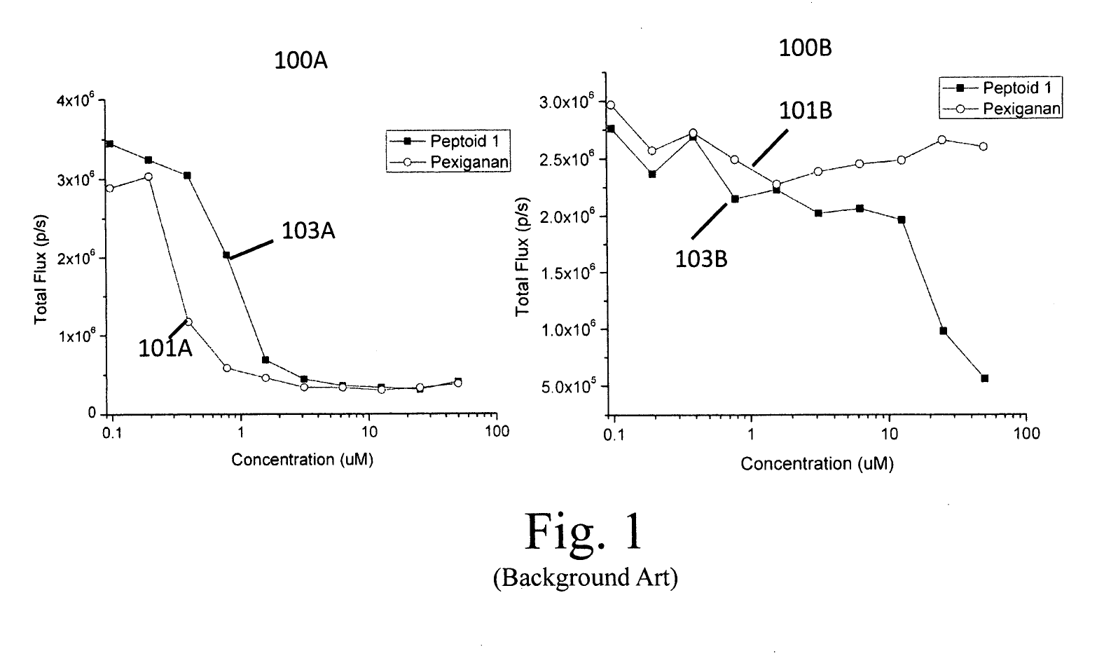

FIG. 2 depicts both a peptide and a peptoid backbone.

FIG. 3 depicts a non-limiting set of n-substituted glycine monomers which may be used to form peptoid polymers having antibacterial activity.

### DETAILED DESCRIPTION OF THE INVENTION

The current invention describes surfaces that are coated with anti-bacterial polymers which comprise one or more n-substituted glycine (peptoid) molecules having antibacterial activity. The surfaces of the current invention can be any surface which is susceptible to bacterial growth, and include, but are not limited to, medical devices such as catheters, stents, cardiovascular implants, biosensors, medical tubing, and implantable electronic devices; consumer goods such as tables, chairs, sunglasses, etc; and other surfaces such as walls, curtains, sinks, etc.

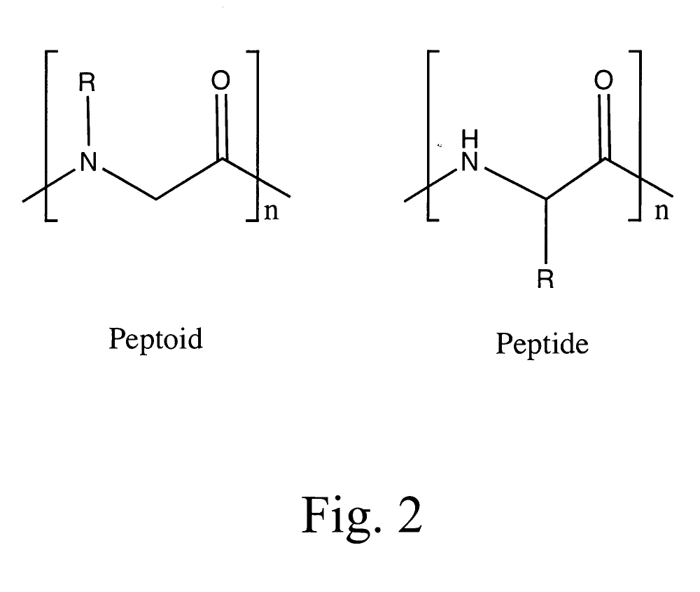

As depicted in FIG. 2, the n-substituted glycines (peptoids) are isomers of amino acids in which the R-group is connected to the amide nitrogen as opposed to the alpha carbon, as is the case for amino acids. The synthesis of peptoids is well established and is described in Zuckermann et al. “Efficient Method for the Preparation of Peptoids [Oligo(N-Substituted glycines)] by Sub-monomer Solid-Phase Synthesis”, J. Amer. Chem. Soc (1992) vol. 114:10646-10647, hereby incorporated by reference as if fully put forth below. The synthesis method is also described in U.S. Pat. Nos. 5,831,005, 5,877,278, and, 5,977,301, hereby incorporated by reference as if fully put forth below. An alternative peptoid synthesis method is described in U.S. Pat. Nos. 5,965,695, and 6,075,121, hereby incorporated by reference as if fully put forth below.

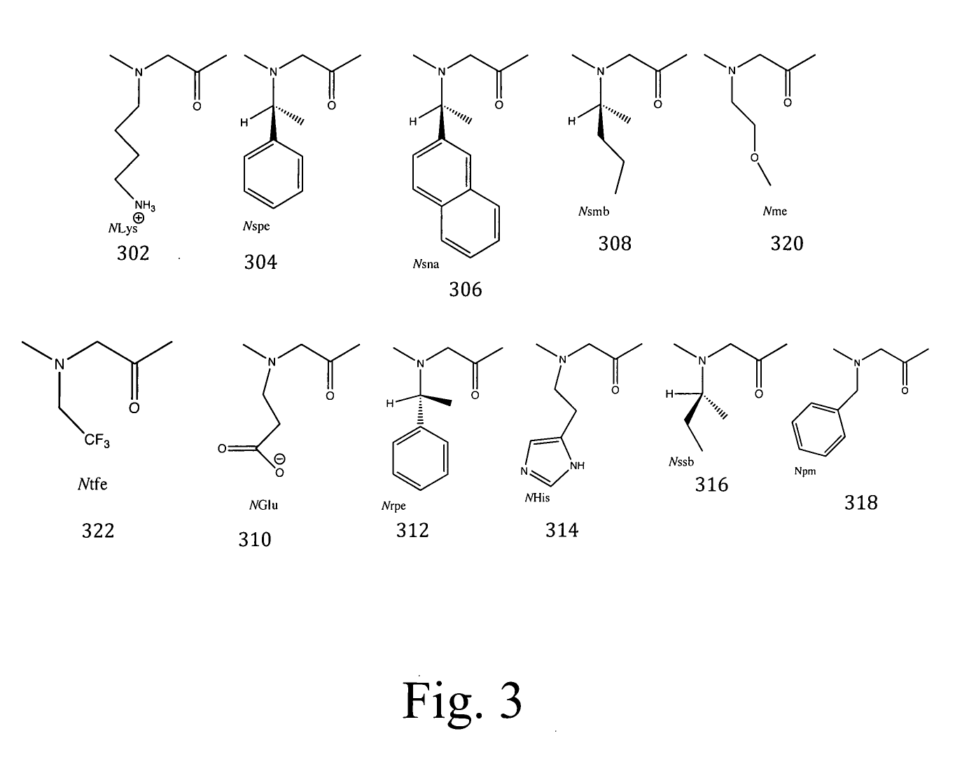

FIG. 3 depicts a non-limiting example of monomers 302-316, which can be used to synthesize peptoid polymers having anti-bacterial activity. In addition to the standard amino acid R-groups such as 302, 310, and 314, other non-standard R-groups such as 304, 306, 308, 312, 316, 318, 320, and 322 can be used. In addition, any primary amine can be used as a peptoid R-group, as is well understood by those of ordinary skill in the art.

The polymer may be comprised completely of peptoid monomer or be chimeric, containing other monomers such as amino acids, or beta amino acids. Accordingly the preferred embodiment of the invention requires that the polymer contain at least one n-substituted glycine and have antibacterial activity (i.e. bactericidal).

In another embodiment of the current invention, the polymer contains at least one n-substituted glycine, and is positively charged and amphiphilic.

The definition of antibacterial activity, for the purposes of the current application, is defined as killing the bacteria (i.e. bactericidal).

The polymers may be covalently attached to the surfaces of the current invention. A non-limiting example of covalently attaching a peptide polymer to a surface is described in U.S. patent application Ser. No. 11/675,500, referred to previously. This method may be extended to the anti-bacterial polymers of the current invention. In addition, the solid phase synthesis method of Zuckermann et al, as described above, may allow for direct covalent synthesis of polymers containing at least one n-substituted glycine, having antibacterial activity, directly on a surface of interest.

In one embodiment of the current invention, the antibacterial polymers of the current invention are attached to a surface via a copolymer that is grafted onto the surface being coated. The copolymer binds the surface through passive physical adsorption rather than covalent chemical reactions. One such polymer, a copolymer of N,N,-Dimethylacrylamide (DMA), N,N,-acryloyloxysuccinimide (NAS) and [3-(meth-acryloyloxy)propyl]trimethoxysilane (MAPS) has been shown to coat silicone (PDMS), a common material used to manufacture urinary tract catheters (e.g. BARD (Lubri-Sil®)). The use and synthesis of copoly(DMA-NAS-MAPS) is described in U.S. patent application Ser. No. 10/536,306, filed on Nov. 29, 2002 and is hereby incorporated by reference as if fully put forth below. Alternative polymers that can be grafted onto a surface and used to conjugate the antibacterial polymers of the current invention are described in U.S. patent application Ser. No. 11/675,500, referred to previously.

An alternative to using copoly(DMA-NAS-MAPS) is to directly functionalize a surface, such as silicone, by exposing the surface to an NH₃ plasma and modifying the surface with star-shaped isocyanate-terminated polyethylene glycol (PEG)-based prepolymers (Star PEG). The antibacterial polymers of the current invention can then be attached directly to the Star PEG. Stat PEG is described in Groll, et al. “A novel star PEG-derived surface coating for specific cell adhesion” J Biomed Mater Res A 2005, 74, (4), 607-17, and is hereby incorporated by reference as if fully put forth below. One advantage of using PEG is that it has been to shown prevent non-specific cell adhesion when covalently attached to surface. Star PEG has been used to covalently link specific cell adhesion peptides to surfaces and this technology can therefore be extended to attach the antibacterial polymers of the current invention to silicone surfaces.

The anti-bacterial polymers of the current invention may be attached to the surface via a linker that comprises 5, 10, 20, or 30 Nme residues and a terminal cysteine residue. Recent work by Statz, et al. “Experimental and theoretical investigation of chain length and surface coverage on fouling of surface grafted polypeptoids” Biointerphases 2009, 4, (2), In Press. demonstrated that a 20 Nme residue linker was sufficient to kill bacteria on the surface of TiO269.

Alternatively, the polymer containing at least one n-substituted glycine having anti-bacterial activity may be non-covalently attached to the surface of the current invention. The peptoid polymer may be applied as a paint or coating independently, or in combination with a resin. Alternatively, a non-covalent attachment scheme, as described in U.S. patent application Ser. No. 11/280,107, filed on Nov. 16, 2005, referred to previously, may be used, in which an adhesive anchor such as a derivative of dihydroxyphenyl (DHPD) is used to adsorb non anti-bacterial N-substituted glycine molecules to a surface.

In an alternative embodiment, the antibacterial polymer could be directly added, without a covalent anchor, as an antibacterial ingredient to paint, resin or cleaning solutions and used to prevent bacterial or fungal growth on a surface treated with paint, resin, or a cleaning solution containing the antibacterial material.

Antibacterial peptoid sequences as depicted by ID 1-15 have been tested for their antibacterial activity. Table 1 lists the minimum inhibitory concentrations (MIC) for E. coli and B. subtilis for ID 1-15. These results are taken from Barron et al. as described above. Table 2 lists the MIC for ID 1 for 5 safety level 2 bacteria demonstrating the broad-spectrum behavior of peptoid antibacterial compounds. The results in Table 2 are taken from Barron et al, as described above.

An alternative to peptoid based anti-bacterial surface coatings is to coat surfaces with peptides and peptoids that have been conformationally constrained by a technique called “hydrocarbon stapling”. This technique constrains the peptide or peptoid to a particular secondary structure resulting in less susceptibility to protease degradation and enhanced uptake by cells. Stapled peptides are discussed in Drahl “Harnessing Helices Chemical braces hold peptides in place, heralding a potential new class of therapeutics” Chemical and Engineering News (2008), Vol 86, No. 22, 18-23 attached as Exhibit J and hereby incorporated by reference as if fully put forth below. Techniques for stapling peptides are described in Schafmeister et al. “An All-Hydrocarbon Cross-Linking System for Enhancing the Helicity and Metabolic Stability of Peptides”, J. Amer. Chem. Soc (2000), 122, 5891-5892, attached as Exhibit K and hereby incorporated by reference as if fully put forth below. Evidence of enhanced activity resulting from stapling peptides is described in Zhang et al. “A Cell-penetrating Helical Peptide as a Potential HIV-1 Inhibitor” J. Mol. Biol. (2008) 378, 565-580, hereby incorporated by reference as if fully put forth below.

### Anti-Bacterial Polymer Synthesis and Characterization

Peptoid synthesis was performed on a custom-built robotic combinatorial synthesizer using the submonomer solid-phase synthesis method described in U.S. Pat. Nos. 5,831,005, 5,877,278, and, 5,977,301, and referred to above. Bromoacetylation of the resin or growing oligomer was followed by displacement of the bromide with a primary amine bearing the desired peptoid sidechain. The N-terminal cysteine monomer was then added using standard Fmoc strategy of solid-phase peptide synthesis (DIC/HOBt). Sidechain deprotecion and cleavage from the resin was accomplished by treating the peptoid with 95:2.5:2.5 TFA:H₂O:TIS for 5 minutes. After lyophilization from acetonitrile:water, the anti-bacterial polymers were purified by prep HPLC (C4 column, 40-80% MeOH w/0.1% TFA) and characterized by analytical HPLC, LC-MS, and MALDI-TOF MS (CHCA or 1,8,9-anthracenetriol matrix).

Rink amide resin (100 mg, 0.6 mmol/g) was swelled in DMF and deprotected (20% 4-methyl piperidine in DMF). A 2-step sequence of bromoacetylation (0.6M bromoacetic acid in DMF, 50% DIC in DMF, 20 min) and amine displacement (1.5M primary amine in NMP, 1.5 h) was carried out at 35° C. for each peptoid monomer. Coupling of the terminal Cys residue was carried out using 0.4M Fmoc-Cys(Trt)-OH and 0.4M HOBt in NMP with 50% DIC in DMF for 2 h at 35° C. After Fmoc removal (20% 4-methyl piperidine in DMF), the remaining protecting groups were removed and the peptoids cleaved from the resin by treatment with 95:2.5:2.5

TFA:H₂O:TIS (5 ml) for 5 min. After filtration to remove the resin, the TFA solution was evaporated under a stream of N2 and the crude peptoids were lyophilized from 50:50 CH₃CN:H₂O (2×30 ml) to give fluffy white solids. Samples were dissolved in 1:1 MeOH:H₂O (10 ml) and purified by prep HPLC (C4 column, 40-80% MeOH w/0.1% TFA, 10 ml/min).

### Anti-Bacterial Polymers Synthesized for Surface Attachment Via Native Chemical Ligation to Polymer:

H-Cys-(Nme)₂₀-(NLys-Nspe-Nspe)₄-NH₂ ID 16

H-Cys-(Nme)₂₀-(NLys-Npm-Npm)₃-NLys-Npm-Nspe-NH₂ ID 17

### Anti-Bacterial Polymers Synthesized for Thiol/Maleimide Attachment:

H—(NLys-Nspe-Nspe)₄-Nte-NH₂, H—(NLys-Npm-Npm)₄-Nte-NH₂ (Nte=thioethylamine) ID 18

HS(CH₂)₂CO—(NLys-Nspe-Nspe)₄-NH₂, HS(CH₂)₂CO—(NLys-Npm-Npm)₄-NH₂ ID 19

### Anti-Bacterial Polymers Synthesized for Solution Testing.

ID 1-19

### Preparation of Silicone Tubing for Surface Attachment of Anti-Bacterial Polymers.

150-1 mm pieces of PDMS tubing (0.38 mm ID×1.98 mm OD) were suspended in a 1% w/v solution of DMA-NAS-MAPS in 20% saturated (NH₄)₂SO₄ (2 ml) with gentle shaking for 30 min. The PDMS pieces were rinsed with H₂O (4×3 ml) and dried under vacuum at 80° C. for 2 h. In a clean vial, the 150 PDMS pieces were suspended in DMF (3.5 ml) along with BnSH (4.1 μL, 10 mM) and iPr₂Net (6.1 μL, 10 mM). The vial was sealed with parafilm and shaken for 2 h. After 22 h at room temperature, the resulting thioester-coated PDMS pieces were removed by filtration and rinsed well with DMF (4×10 ml). Cys-peptoid (3 mg) and TCEP (30 mg, 40 mM) were dissolved in pH 7 phosphate buffer (2.5 ml) and DMF (0.5 ml) and incubated at 40° C. for 2 h. The thioester-coated PDMS pieces and thiophenol (2% v/v, 60 μl) were added to the peptoid solution. The vial was sealed with parafilm, shaken well, and left to stand at room temperature for 22 h. The resulting peptoid-coated PDMS pieces were removed, rinsed well with DMF (4×10 ml), and dried by suction filtration.

### Attachment of Anti-Bacterial Polymers to Silicone

The terminal cysteine residues were used to conjugate the antibacterial peptoid to the NHS-ester of the copoly(DMA-NAS-MAPS) polymer using a native chemical ligation scheme. The NHS ester of the polymer is first converted to a benzyl thioester using BnSH (benzyl mercaptan) and DIPEA (diisopropylethylamine). The thioester-containing polymer can then undergo a native chemical ligation reaction with the N-terminal Cys of the peptoid. In this reaction, transesterification occurs when the thiol of the cysteine residue attacks the thioester. This is followed by a 5-member ring rearrangement to form a stable amide bond with the cysteine α-amino group. As a test reaction, Fmoc-Gly-OSu (the NHS ester of Fmoc-glycine) is used in place of the polymer and converted to a benzyl thioester (Fmoc-Gly-SBn), which is then subjected to a competition reaction with cysteine, alanine, and (β-ala-NH₂.HCl in pH 7 phosphate buffer and DMF. Alanine was included in the reaction to verify that the thiol sidechain is necessary for reactivity. Beta-alanine amide hydrochloride is included to mimic the primary amine sidechains of the anti-bacterial polymer and to verify that they do not interfere with the reaction. After stirring overnight at room temperature, the only product detected by LC/MS had a mass corresponding to the expected Fmoc-Gly-Cys-OH dipeptide. Prior to surface attachments, the anti-bacterial polymer is incubated with TCEP in pH 7 buffer and DMF to reduce any disulfide bonds formed during purification and storage. The anti-bacterial polymer containing solution is then exposed to the thioester-containing polymer-coated surface. Thiophenol (2% v/v) is added to keep the thiols of the antibacterial reduced and to regenerate any unproductive thioesters that are formed. After 24 hours at room temperature, the coated surface is rinsed well with DMF and analyzed.

### Characterization of DMA-NAS-MAPS Copolymer Coated Surface.

After coating the desired surface with the DMA-NAS-MAPS copolymer, the number of NHS-esters available for conjugation to the antibacterial peptoids were measured using an NHS-ester hydrolysis assay. Briefly, the NHS esters were hydrolyzed in the presence of 0.1N NH₄OH (2-20 min) to generate the hydroxysuccinimide anion, which has an absorption peak at 260 nm. The absorbance was measured and the extinction coefficient of 9700 M⁻¹ cm⁻¹ was used to calculate the actual concentration of NHS esters in the sample. Preliminary samples gave a measured NHS content of 1.44×10¹⁴ esters/cm².

### Determination of Surface Coverage of Conjugated Anti-Bacterial Polymers.

50-1 mm pieces of PDMS coated with copoly DMA-NAS-MAPS were suspended in 250 μl of 0.1N NH₄OH and let stand 15 min with occasional shaking. A 100 μl aliquot was removed and its absorbance measured at 260 nm. The measured absorbance of 0.056 corresponds to a sample concentration of 5.8 μM. Based on a surface area of 0.121 cm² for each 1 mm piece of PDMS, this is equal to a copolymer surface coverage of 1.44×10¹⁴ NHS esters/cm².

The number of anti-bacterial polymers that are conjugated to the surface was determined by reacting the free amines of the NLys residues with Fmoc-Cl. The Fmoc groups was then released by treatment with 20% 4-methyl piperidine in DMF, and the absorbance of the resulting solution was measured at 301 nm (extinction coefficient=6000 M⁻¹ cm⁻¹). For two different anti-bacterial polymer sequences (i.e. H-Cys-(Nme)₂₀-(NLys-Nspe-Nspe)₄-NH₂, and H-Cys-(Nme)₂₀-(NLys-Npm-Npm)₃-NLys-Npm-Nspe-NH₂), the surface coverage was measured to be about 2.86×10¹³ peptoids/cm² (20% of available NHS esters reacted).

The Fmoc-Cl (3.6 mg) and iPr₂Net (2.4 μL) were dissolved in CH₂Cl₂ (1 ml). The Fmoc-Cl solution (500 μl) was added to 50 peptoid-coated PDMS pieces and shaken for 1 h at room temperature. The PDMS pieces were filtered, rinsed well with CH₂Cl₂ (20 ml) and DMF (10 ml), and transferred to a clean vial. 20% 4-methyl piperidine in DMF (500 μL) was added to the PDMS pieces and shaken for 20 min. A 100 μl aliquot was removed and its absorbance measured at 301 nm. The measured absorbance of 0.759, minus the absorbance (0.745) of a control set of thioester-coated PDMS pieces treated in the same manner, corresponds to a sample Fmoc concentration of 2.3 μM. Based on a surface area of 0.121 cm² for each 1 mm piece of PDMS and the 4:1 amine:peptoid ratio, this is equal to a surface coverage of 2.86×10¹³ peptoids/cm².

### Testing Anti-Bacterial Coated Silicone Surfaces for Antibacterial Activity

Antibacterial activity is assessed for uropathogenic gram-negative E. coli (ATCC 35218) and gram-positive B. subtilis (ATCC 6633). Bacteria is grown at 37° C. with shaking at 180 rpm in Luria (LB) broth (Sigma) to the mid-log phase as determined by the optical density at 600 nm. The bioassays with anti-bacterial polymer covalently attached to grafted copoly(DMA-NAS-MAPS) to silicone tubing is done in culture tubes. Appropriate amounts of 2-4 cm long anti-bacterial coated silicone tubing is added to the culture tubes containing 1 ml of LB medium. Then, an aliquot of the cell suspension is added, resulting in a cell concentration of about 1.6×106 cells/ml. Uncoated silicone and copoly(DMA-NAS-MAPS)-coated silicone is used as a control. To evaluate the antimicrobial activity of the anti-bacterial polymer coated silicone, we shake the test tubes horizontally to enhance the probability of cell-PDMS contact. After about 17 h of exposure, the absorbance at 600 nm is measured and used to quantify the number of cells in the culture tubes.

Peptoid ID Listing

ID 1: H—(NLys-Nspe-Nspe)₄-NH₂

ID 2: H—(NLys-Nssb-Nspe)₄-NH₂

ID 3: H—(NLys-Nrpe-Nrpe)₄-NH₂

ID 4: H—(NLys-Nspe-Nspe)₂-NH₂

ID 5: H—(NLys-Nspe-Nspe)₃-NH₂

ID 6: H—(NLys-Nspe-Nspe)₅-NH₂

ID 7: H—(NLys-Nsmb-Nspe)₄-NH₂

ID 8: H—(NLys-Nssb-Nspe-NLys-Nssb-Nsna)₂-NH₂

ID 9: H—(NLys-Nspe-Nspe-NLys-Nspe-Nsna)₂-NH₂

ID 10: H—(NLys-Nspe-Nspe-NLys-Nspe-NHis)₂-NH₂

ID 11: H—(NLys-Nspe-Nspe-NLys-Nspe-L-Pro-(NLys-Nspe-Nspe)₂-NH₂

ID 12: H—(NLys-Nspe-Nspe-NGlu-Nspe-Nspe)₂-NH₂

ID 13: H—(NGlu-Nspe-Nspe)₄-NH₂

ID 14: H—(NLys)₄-(Nspe)₈-NH₂

ID 15: H-NLys-Nssb-Nspe-Nssb-Nspe-NLys-Nspe-NLys-Nssb-Nssb-Nspe-NLys-NH₂

ID 16: H-Cys-(Nme)₂₀-(NLys-Nspe-Nspe)₄-NH₂

ID 17: H-Cys-(Nme)₂₀-(NLys-Npm-Npm)₃-NLys-Npm-Nspe-NH₂

ID 18 H—(NLys-Nspe-Nspe)₄-Nte-NH₂, H—(NLys-Npm-Npm)₄-Nte-NH₂ (Nte=thioethylamine)

ID 19 HS—(CH₂)₂CO—(NLys-Nspe-Nspe)₄-NH₂, HS(CH₂)₂CO—(NLys-Npm-Npm)₄-NH₂

TABLE 1

| | E. coli | B. subtilis |
|---|---|---|
| ID | MIC (uM) | MIC (uM) |
| 1 | 3.5 | .88 |
| 2 | 31 | 3.9 |
| 3 | 3.5 | .88 |
| 4 | 27 | 27 |
| 5 | 9.1 | 1.2 |
| 6 | 5.5 | 1.4 |
| 7 | 7.4 | .95 |
| 8 | 7.2 | .93 |
| 9 | 3.3 | 1.6 |
| 10 | 3.5 | 6.9 |
| 11 | 3.1 | 1.6 |
| 12 | >110 | 6.9 |
| 13 | >219 | >219 |
| 14 | 6.9 | 1.7 |
| 15 | 3.1 | 15 |

TABLE 2

| | ID 1 | Pexiganan |
|---|---|---|
| Bacteria | MIC (uM) | MIC (uM) |
| Streptococcus pneumoniae | 1.7-3.4 | 13 |
| Haemophilus influenzae | 6.9 | 3.2 |
| Staphylococcus aureus | 3.4 | 6.5-13 |
| Escherichia coli | 14-28 | 3.2-6.5 |
| Enterococcus faecalis | 3.4-6.9 | 26 |
| Psuedomonas aeruginosa | 28 | 3.2-6.5 |

What we claim:

1. A surface coated with an anti-bacterial molecule, wherein the anti-bacterial molecule is a polymer comprising one or more n-substituted glycine monomers, and wherein the polymer shows anti-bacterial activity.

2. The surface according to claim 1, wherein the polymer is a chimeric polymer comprising amino-acid and n-substituted glycine monomers.

3. The surface according to claim 1, wherein the polymer is covalently attached to the surface.

4. The surface according to claim 1, wherein the polymer is non-covalently attached to the surface.

5. The surface according to claim 1, wherein the polymer is positively charged and amphiphilic.

6. The surface according to claim 1, wherein the polymer is selected from group comprising ID 1-19.

7. A method for creating a surface having anti-bacterial activity, comprising applying a polymer to the surface, wherein the polymer includes at least one n-substituted glycine monomer and wherein the polymer has anti-bacterial activity.

8. The method according to claim 7, where applying the polymer comprises covalently attaching the polymer to the surface.

9. The method according to claim 7, where applying the polymer comprises non-covalently attaching the polymer to the surface.

10. The method according to claim 7, wherein the polymer is selected from a group comprising ID 1-19.
